(19) United States
(12) Patent Application Publication
(10) Pub. No.: US 2025/0099385 A1
(43) Pub. Date: Mar. 27, 2025

(54) Extended Release Boswellic Acid and Manufacturing Thereof

(71) Applicant: Lonza Greenwood LLC, Greenwood, SC (US)

(72) Inventors: Jowell Bolivar, Greenwood, SC (US); Shane Durkee, Greenwood, SC (US); Tyler White, Greenwood, SC (US); Zainulabedin Saiyed, Benicia, CA (US)

(21) Appl. No.: 18/834,006

(22) PCT Filed: Jan. 31, 2023
(86) PCT No.: PCT/US2023/011978

§ 371 Date: Jul. 29, 2024

Related U.S. Application Data
(60) Provisional application No. 63/305,922, filed on Feb. 2, 2022.

(51) Int. Cl. A61K 9/16; A61K 9/00; A61K 31/22; A61K 45/06; A61P 29/00

(52) U.S. Cl. CPC A61K 9/1617; A61K 9/0002; A61K 9/0053; A61K 31/22; A61K 45/06; A61P 29/00

(57) ABSTRACT

The present disclosure is directed to an extended release composition containing one or more Boswellia derived compounds in a lipid matrix, which releases the Boswellia derived compound over a period of time. In one embodiment, the composition contains an Boswellic acid compound. In a particular embodiments the Boswellic acid compound is 3-O-acetyl-11-keto-.beta.-boswellic acid (AKBA).

This application is based on and claims priority to U.S. Provisional Application No. 63/305,922, filed on Feb. 2, 2022, which is expressly incorporated herein by reference in its entirety.

### BACKGROUND

The resin of Boswellia species has been used as incense in religious and cultural ceremonies and in medicines and herbal remedies, including as an anti-inflammatory, for centuries. Boswellia serrata is a moderate to large sized branching tree of family Burseraceae (Genus Boswellia), grows in dry mountainous regions of India, Northern Africa and Middle East. Oleo gum-resin is tapped from the incision made on the trunk of the tree and is then stored for a period of time to remove the oil content, resulting in the resin being solidified. Boswellia resin and extracts of the resins have been in use as dietary supplements for the treatment of inflammatory conditions for many years. In particular, Boswellic acids were identified as the active compounds responsible for the beneficial effects attributed to Boswellia resins and extracts. Of the boswellic acids, the boswellic acid compound 3-O-acetyl-11-keto-.beta.-boswellic acid (AKBA) was identified as the most potent 5-lipoxygenase inhibitor of all the boswellic acids and therefore has the best anti-inflammatory benefits [Sailer E R et. al., British J Pharmacology. 1996, 117(4): 615-8].

Boswellic acids, including 3-O-acetyl-11-keto-.beta.-boswellic acid (AKBA) have been blended with other ingredients as a dietary supplement for the prevention, control, or treatment of inflammation, including joint inflammation, such as knees, hips, shoulders, elbows, and wrist. The problem with Boswellia acid, in particular, AKBA, is that AKBA is a highly water-insoluble compound and is poorly bioavailable, and generally has very short therapeutic window. Studies showed that AKBA levels in the blood normally peaks at almost 3 hours after oral consumption, and the AKBA levels are at a nearly zero concentration beyond 10 hours from oral consumption. Hence, in order for Boswellic acid compound, in particular AKBA, to be effective, the Boswellic acid compound must be administered to the mammal in need of treatment more than once-a-day. However, many other anti-inflammation compound or joint pain relieving compound are administered only once a day which is often found to be convenient for users of dietary supplements.

Further, it is generally known that various Boswellia resins extracts, including AKBA, tend to have a poor bioavailability. It is believed this is caused by poor aqueous solubility of the Boswellia extracts and Boswellic acids.

There is a need in the art to have a Boswellic acid compound delivered to a mammal over a period of time so that the Boswellic acid compound can be administered once a day. In addition, there is a need in the art to have a way for the Boswellia extracts, including Boswellic acids, such as AKBA, to be made available for the body to process these Boswellia derived compounds to obtain the benefit of anti-inflammation. The present disclosure provides a solution to that need.

### SUMMARY

In general, the present disclosure is directed to an extended release composition containing at least one Boswellia derived compound, such as a Boswellia extract or Boswellia compound, such as Boswellic acid, where the Boswellia derived compound is released from the composition over a period of time. In one aspect, for instance, the present disclosure is directed to a dietary supplement composition in which amounts of Boswellia derived compound are contained or dispersed within an edible lipid system that is capable of delivering effective amounts of Boswellia derived compound to a mammal for various other health benefits, including reduced inflammation. In a particular aspect of the disclosure, the edible lipid system is a lipid multiparticulate. In addition, through the methods and compositions of the present disclosure, the bioavailability of a Boswellia derivative compound can be greatly enhanced in a mammal.

In another aspect of the present disclosure, the active agent Boswellia derivative comprises a Boswellic acid compound. A particular the Boswellia acid compound comprises 3-O-acetyl-11-keto-.beta.-boswellic acid (AKBA).

In a further aspect of the present disclosure, the active agent is released from the composition over a period up to about 30 hours after ingestion by a user, such as in period of time from about 0.5 hours to 24 hours after ingestion, and more particularly in a period of time from about 1 hour to about 20 hours after ingestion.

Another feature of the present disclosure, the active agent is encapsulated by the lipid matrix. Further, the active agent is present in the lipid multiparticulate particles in an amount from about 1% to about 80% by weight, such as in an amount from about 10% to about 75% by weight, more particularly in an amount from about 25% to about 70% by weight based on the total weight of the lipid multiparticulate particles. The lipid multiparticulate particles have an average particle size of greater than 1 μm, generally greater than 10 μm, typically from about 40 microns to about 3000 microns, such as from 100 microns to 2000 microns.

In one particular aspect of the present disclosure, the lipid matrix contains at least one low flow point excipient and at least one high flow point excipient. Typically the low flow point excipients are present in the composition in an amount of from about 0.1% to about 20% by weight and wherein the high flow point excipients are present in the composition in an amount of from about 20% to about 85% by weight based on the total weight of the composition

In a further embodiment of the present disclosure, the lipid matrix contains a fatty alcohol, a fatty acid, a fatty acid ester of a glycol and a poly glycol, a fatty acid ester of glycerol, polyglycerol, a polyglycolized glyceride, a C10-C18 triglycerides stearoyl polyoxylglyceride, a lauroyl macrogol-32 glyceride, a caprylocaproyl macrogol-8 glyceride, an oleoyl macrogol-6 glyceride, a linoleoyl macrogol-6 glyceride, myristyl alcohol, lauryl alcohol, capric alcohol, glycerol behenate, glycerol dibehenate, glycerol palmitate, hydrogenated castor oil, stearyl alcohol, behenyl alcohol, palmitic acid, stearic acid, paraffin wax, beeswax, candelilla wax, carnauba wax, polyethoxylated 12-hydroxysteric acid, a propylene glycol fatty acid ester, esterified alpha-tocopheryl polyethylene glycol succinate, a propylene glycol monolaurate (C12) ester, polyoxyl 35 castor oil, polyoxyl 40 hydrogenated castor oil, a lecithin, vitamin E, tocopheryl polyethylene glycol succinate (TPGS), a sugar fatty acid ester, a sorbitan fatty acid ester, a polyoxyethylene sorbitan fatty acid ester, a polyoxyethylene-polyoxypropylene copolymer, rosemary extract, propylene glycol, triacetin, isopropyl myristate, diethylene glycol monoethyl ether, polyethylene glycol, glycerol, mixtures or combinations thereof.

In another embodiment of the present disclosure, the lipid matrix contains a wax, a fatty alcohol, and a fatty acid. In a particular embodiment, the wax comprises candelilla wax, wherein the fatty alcohol comprises stearyl alcohol, and wherein the fatty acid comprises stearic acid.

In an further embodiment, the lipid matrix may further contain a surfactant. Suitable surfactants include, for example, polysorbate, a laureth sulfate, or mixtures thereof.

The lipid matrix may further contain other additional ingredients, such as flow aids, antioxidant, dispersing agent and/or a flavoring or sweetener.

In an aspect of the present disclosure, the extended release composition, including the particles, may be placed into a capsule, formed into a tablet, placed in a soft-gel, placed in a gummy, may be alternatively ingested directly by a mammal as a powder or can be incorporated into a beverage or other food item.

In another embodiment of the present disclosure, provided is a method for administering a Boswellia derived compound to a mammal over an extended period of time. The method comprising orally administering to a mammal an extended release composition having lipid multiparticulate particles, the lipid multiparticulate particles containing a lipid matrix. The active agent is dispersed in the lipid matrix and wherein the active agent comprising a Boswellia derived compound. Each dosage administered to the mammal containing the Boswellia derived compound in an amount from about 1 mg to about 1,000 mg, for example, 2 mg to about 500 mg and more particularly between about 5 mg to 200 mg.

In a further embodiment of the present disclosure, provided is a method where the extended release composition is formulated such that Boswellia derived active agent is released from the extended release composition over a period up to about 30 hours after oral administration to a user, such as in period of time from about 0.5 hours to 24 hours after administration to a user, and more particularly in a period of time from about 1 hour to about 20 hours after administration to a user.

In a further embodiment of the present disclosure, provided is method of reducing inflammation in a mammal, said method comprising providing the extended release composition described above in any one of the previous aspects and embodiments of the present disclosure and administering the extended release composition to the mammal in need of a reduction in inflammation.

In a yet a further embodiment provided is method of method of increasing the bioavailability of a Boswellia derived compound in a mammal said method comprises forming an extended release composition described above in any one of the previous aspects and embodiments of the present disclosure and administering the extended release composition to the mammal.

In another embodiment of the present disclosure, provided is a nutraceutical composition containing the extended release composition described above in any one of the previous aspects and embodiments of the present disclosure and a second nutraceutical ingredient. One example of a second nutraceutical ingredient comprises undenatured collagen.

Other features and aspects of the present disclosure are discussed in greater detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figures in which:

FIG. 1 is a graphical illustration of the extended release dissolution profiles for AKBA from different lipid multiparticulate in Example 3;

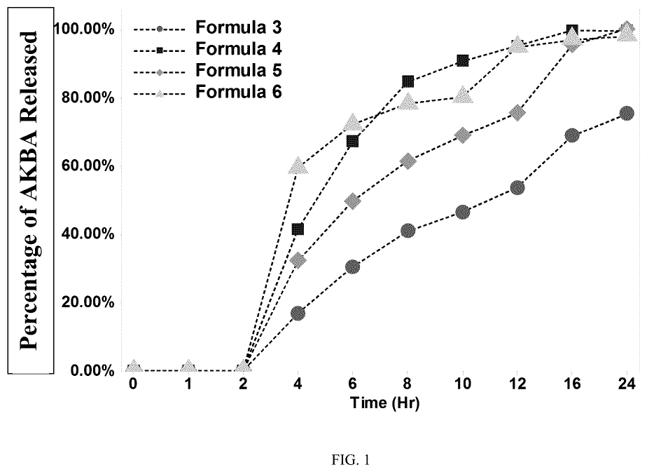

FIG. 2 is a graphical illustration of the reduction in OA disease severity measured by radiography according to Example 4.

FIG. 3 is a graphical illustration of the increase in weight bearing according to Example 4.

FIG. 4 is a graphical illustration of the reduction in knee swelling according to Example 4.

FIGS. 5A and 5B are graphical illustrations of the reduction in inflammatory markers according to Example 4.

FIGS. 6A and 6B is graphical illustrations of the reduction in cartilage breakdown markers according to Example 4.

Repeat use of reference characters in the present specification and drawings is intended to represent same or analogous features or elements of the invention.

### Definitions

As used in this application and in the claims, the singular forms “a,” “an,” and “the” include the plural forms unless the context clearly dictates otherwise. Additionally, the term “includes” means “comprises.” The methods and compositions of the present disclosure, including components thereof, can comprise, consist of, or consist essentially of the essential elements and limitations of the embodiments described herein, as well as any additional or optional ingredients, components or limitations described herein or otherwise useful in nutritional compositions.

Unless otherwise indicated, all numbers expressing quantities of ingredients, properties such as molecular weight, percentages, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited. As used herein, the terms “about,” “approximately,” or “generally,” when used to modify a value, indicates that the value can be raised or lowered by 10%, such as, such as 7.5%, 5%, such as 4%, such as 3%, such as 2%, such as 1%, and remain within the disclosed aspect. Moreover, the term “substantially free of” when used to describe the amount of substance in a material is not to be limited to entirely or completely free of and may correspond to a lack of any appreciable or detectable amount of the recited substance in the material. Thus, e.g., a material is “substantially free of” a substance when the amount of the substance in the material is less than the precision of an industry-accepted instrument or test for measuring the amount of the substance in the material. In certain example embodiments, a material may be “substantially free of” a substance when the amount of the substance in the material is less than 10%, less than 9%, less than 8%, less than 7%, less than 6%, less than 5%, less than 4%, less than 3%, less than 2%, less than 1%, less than 0.5%, or less than 0.1% by weight of the material.

As used herein, “optional” or “optionally” means that the subsequently described material, event or circumstance may or may not be present or occur, and that the description includes instances where the material, event or circumstance is present or occurs and instances in which it does not. As used herein, “w/w %” and “wt %” means by weight as a percentage of the total weight or relative to another component in the composition.

The phrase “effective amount” means an amount of a compound that promotes, improves, stimulates, or encourages a response to the particular condition or disorder or the particular symptom of the condition or disorder.

The term “therapeutically effective amount” as used herein, shall mean that dosage, or amount of a composition, that provides the specific pharmacological or nutritional response for which the composition is administered or delivered to mammals in need of such treatment. It is emphasized that “therapeutically effective amount”, administered to a particular subject in a particular instance, will not always be effective in treating the ailments or otherwise improve health as described herein, even though such dosage is deemed a “therapeutically effective amount” by those skilled in the art. Specific subjects may, in fact, be “refractory” to a “therapeutically effective amount”. For example, a refractory subject may have a low bioavailability or genetic variability in a specific receptor, a metabolic pathway, or a response capacity such that clinical efficacy is not obtainable. It is to be further understood that the composition, or supplement, in particular instances, can be measured as oral dosages, or with reference to ingredient levels that can be measured in blood. In other embodiments, dosages can be measured in amounts applied to the skin when the composition is contained with a topical formulation.

The term “nutraceutical” and refers to any compound added to a dietary source (e.g., a food, beverage, or a dietary supplement) that provides health or medical benefits in addition to its basic nutritional value.

The term “delivering” or “administering” as used herein, refers to any route for providing the composition, product, or a nutraceutical, to a subject as accepted as standard by the medical community. For example, the present disclosure contemplates routes of delivering or administering that include oral ingestion plus any other suitable route of delivery including transdermal, intravenous, intraperitoneal, intramuscular, topical and subcutaneous.

As used herein, the term “mammal” includes any mammal that may benefit from improved joint health, resilience, and recovery, and can include without limitation human, canine, equine, feline, bovine, ovine, or porcine mammals. For purposes of this application, “mammal” does include human subjects.

The term “supplement” means a product in addition to the normal diet but may be combined with a mammal's normal food or drink composition. The supplement may be in any form but not limited to a solid, liquid, gel, capsule, or powder. A supplement may also be administered simultaneously with or as a component of a food composition which may comprise a food product, a beverage, a pet food, a snack, or a treat. In one embodiment, the beverage may be an activity drink.

As used herein, “healthy” refers to the absence of illness or injury.

As used herein, the term “flow point” is the temperature at which any portion of the mixture becomes sufficiently fluid that the mixture, as a whole, may be atomized. Generally, a mixture is sufficiently fluid for atomization when the viscosity of the molten mixture is less than 20,000 cp, or less than 15,000 cp, or less than 10,000 cp, less than 5000 cp, or even less than 1000 cp. The viscosity can be measured by a controlled stress rheometer, which measures viscosity as a function of temperature, and may use either a shear-type or rotational rheometer. As used herein, melting point refers to the temperature that marks the midpoint of the transition from a solid crystalline or semi-crystalline state to a liquid state. As measured by DSC, the melting point is the temperature where upon heating the solid material, the maximum exothermic heat flow occurs. In general, melting point will be used in reference to relative pure single component materials such as some actives or essentially single component excipients (e.g. stearyl alcohol) and flow point will be used in reference to multi-component materials or mixtures.

As used herein, the term “semi-solid” is a solid at ambient temperature (23° C.) but becomes a liquid at temperatures above 30° C. or 40° C., or at body temperature.

Unless otherwise indicated, “capsule” means a container suitable for enclosing solids or liquids and includes empty capsule shells and components thereof such as caps and bodies that may be assembled together to form the capsule.

As used herein, by “active” or “active ingredient” is meant a drug, medicament, pharmaceutical, therapeutic agent, nutraceutical, or other compound that may be desired to be administered to the body. The active ingredient may be a “small molecule,” generally having a molecular weight of 2000 Daltons or less. The active ingredient may also be a “biological active.” Biological active ingredients include proteins, antibodies, antibody fragments, peptides, oligonucleotides, vaccines, and various derivatives of such materials. In one embodiment, the active ingredient is a small molecule. In another embodiment, the active ingredient is a biological active. In still another embodiment, the active ingredient is a mixture of a small molecule and a biological active. Also as used herein, the terms “active ingredient”, “first active ingredient”, “second active ingredient”, etc. may be used to denote active ingredients located in different places within the particle, such as those located in the core or those located in the one or more outer layers. However, the terms “first” or “second” do not necessarily denote that the first active ingredient is different from the second active ingredient. For example, in certain embodiments, the active ingredient contained within the core may be the same as the second active ingredient contained within an outer layer disposed on the core. While in certain other embodiments, the active ingredient contained within the core may be different from the second active ingredient contained within an outer layer disposed on the core

Unless otherwise indicated, “dosage form” refers to a solid composition comprising an active ingredient.

As used herein, the term “particle” refers a portion or quantity of material(s), such as a small portion or quantity of material(s). For example, as provided herein, the term particle may refer generally to a composition containing a core and one or more outer layers surrounding the core. In some embodiments, the particle(s) described may be generally spherical in shape. The term “particle” as used herein includes or may be used interchangeably with the following: pellet, beadlet, multiparticulates, particulates, spheres, including microspheres, seeds, and the like. The term particle as used herein is not limited to only a particle formed by certain methods or processes. Indeed, the particle(s) described herein may be formed by any suitable process. Certain suitable processes include, but are not limited to, melt spray congealing, spheronization, extrusion, compression, powder layering, liquid layering, pelletization by melt and wet granulation, and combinations thereof. The particle(s) as described herein may be solid or semi-solid particles. In some embodiments, the particles describe herein can include both solid and semi-solid compositions contained on or within the particle itself.

As used herein, percent change in any one or more of the discussed biomarkers is based upon a baseline value for the respective subject and/or marker and does not refer to a percent change from a midpoint or non-starting point. Therefore, as used herein, “baseline” refers to a day zero or starting point, and can therefore be used to determine a value of a measured marker at a starting point, which is then used to calculate the percent change in any of the biomarkers discussed herein from the baseline.

Other features and aspects of the present disclosure are discussed in greater detail below.

### DETAILED DESCRIPTION

It is to be understood by one of ordinary skill in the art that the present discussion is a description of exemplary embodiments only, and is not intended as limiting the broader aspects of the present disclosure.

The present disclosure is generally directed to lipid multiparticulates containing a Boswellia derivative compound. The particles, may be placed into a capsule, formed into a tablet, placed in a soft-gel, placed in a gummy, may be alternatively ingested directly by a mammal as a powder or can be incorporated into a beverage or other food item. The lipid multiparticulate particles include a lipid matrix that, in one embodiment, can be formulated to release the Boswellia derivative compound when the particles are in contact within an environment which cause the Boswellia derivative compound to be released from the lipid multiparticulates, such as in the digestive systems of a mammal that has been orally administered or otherwise ingested the lipid multiparticulates.

The following description is exemplary in nature and is not intended to limit the scope, applicability or configuration of the invention in any way. Various changes to the described embodiments may be made in the function and arrangement of the elements described herein without departing from the scope of the disclosure.

Embodiments of the disclosed composition may include at least one active ingredient or active agent. The compositions may contain one or more active ingredients. As described above, in one embodiment, the active ingredient can be one or more Boswellia derivative compounds which are incorporated or dispersed into a lipid matrix. In one embodiment, the composition of the present disclosure extended release composition comprising a lipid multiparticulate that delays the release of one or more Boswellia derivative compounds beyond the initial time when the extended release composition enters the digestive system of a mammal, such as a human, to deliver a fairly constant dose of the Boswellia derivative compounds to the mammal over a period of time. For example, the one or more Boswellia derivative compounds can be dispersed or encapsulated within a lipid matrix that is specially formulated to entrap the one or more Boswellia derivative compounds and postpone their release from the lipid matrix for a period of time. Of particular advantage, the particles of the present disclosure can be constructed to be 100% vegetarian. In addition, the particle size can be carefully controlled and adjusted to fit different purposes, such as when producing capsules, beverages, tablets, and the like.

Moreover, in addition to the beneficial extended release properties, the present disclosure has also surprisingly found that the extended release composition could reduce pro-inflammatory cytokines, reduce pain, and reduce inflammation, including subjective reduction in inflammation and/or joint pain, or combinations thereof, even at low dosages and administration time periods where the anti-inflammatory compound is present in the form of a lipid multiparticulate. Namely, as will be discussed in greater detail below, the present disclosure has found that certain anti-inflammatory compounds in combination with collagen, and in a lipid multiparticulate dosage form in combination with collagen allows for increased bioavailability and nutrient efficacy without requirement of large dosages or extensive administration times.

The extended release composition of the present disclosure, when administered to mammals suffering from joint pain and/or inflammation, can reduce joint pain and inflammation in skeletal joints. When administered to mammals suffering from joint pain and/or inflammation, the extended release composition can dramatically decrease serum 5-lipoxygenase (5-LOX) protein levels in a mammal suffering from joint pain and/or inflammation. 5-LOX levels can decrease by greater than about 10%, such as greater than about 20%, such as greater than about 30%, such as greater than about 40%, and generally less than about 50%. The above reductions can occur after a period of time of being administered the extended release composition at least once every three days, such as at least every day. Moreover, as discussed above, the above results also occur more quickly than previously compositions and are therefore considered to be “fast acting”. Thus, in one aspect, the dramatic reduction in inflammation and joint pain, for instance, can also be exhibited even after only two weeks or less of administration, such as after only 10 days or less of administration, such as after only 1 week or less of administration, such as after only 5 days or less of administration, such as after as little as 3 days or less of administration. However, it should be understood that the extended release composition of the present disclosure also provides long-term benefits, as the above, or any of the following benefits are also exhibited over the course of supplementation with the extended release composition. Thus, in one aspect, the reduction in inflammation and joint pain for any of the biomarkers discussed herein are exhibited for about 2 weeks or more, such as about 1 month or more, such as about 6 weeks or more, or for any of the supplementation times discussed herein.

As shown above, the effect on biomarkers within the mammal can be dramatic and far exceed biomarker influence recorded in the past when a mammal has been administered collagen or an anti-inflammatory agent alone, or without the anti-inflammatory agent being in the delivery form of a lipid multiparticulate. Furthermore, as discussed above, the joint-health composition of the present disclosure exhibits an improvement in joint health or reduced inflammation as evidenced by one or more of the above factors more quickly and/or effectively than previous supplements. Thus, as discussed above, the joint-health composition of the present disclosure can be considered to be “fast acting” and exhibit improvements in as little as two weeks of administration, such as after only 10 days or less of administration, such as after only 1 week or less of administration, such as after only 5 days or less of administration, such as after as little as 3 days or less of administration. Furthermore, as may be discussed in greater detail below, it should be understood based upon the discussion of the benefits of the extended release composition of the present disclosure, that bioavailability of the components of the extended release composition is unexpectedly improved, including where the extended release composition is tailored for delayed release, sustained release, or steady release formulations. A “delayed release” means that release of the active is shifted later in time, based on time alone or based on other conditions, such as pH, temperature and the like. “Sustained release” means are release of the active over a length of time, such as 2 hours, 4, hours, 8 hours, 12 hours or 24 hours, or any time interval in between once the release of the active has begun. A “steady release” means a release of the active at a nearly constant rate once the release of the active has started.

However, as noted above, in one aspect, it should be understood that the reduction joint pain and/or inflammation is a subjective perception by the mammal of an improvement in joint pain and/or inflammation, and does not require measurement of one or more inflammatory markers.

As used herein, the term “Boswellia derivative compound” is defined as the Boswellia resin itself and extracts from the resin. The resinous part of Boswellia serrata possesses monoterpenes, diterpenes, triterpenes, tetracyclic tri-terpenic acids and four major pentacyclic triterpenic acids, which are beta-boswellic acid, acetyl-beta-boswellic acid, 11-keto-beta-boswellic acid and acetyl-11-keto-beta-boswellic acid. These compounds separately or together are considered herein as being a Boswellia derivative compound. It is believed that these compounds are responsible for inhibition of pro-inflammatory enzymes. Of particular importance is the Boswellic acid compounds. Out of these four boswellic acids, 3-O-acetyl-11-keto-.beta.-boswellic acid (AKBA) which has the structure (1):

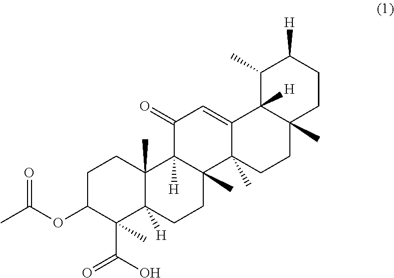

is recognized as the most potent inhibitor of 5-lipoxygenase, an enzyme responsible for inflammation. AKBA is a highly water-insoluble compound and generally has short therapeutic window. Studies showed that AKBA levels in the blood normally peaks at almost 3 hours after oral consumption, and AKBA has almost zero concentrations beyond 10 hours from several tests.

In the present disclosure, using Boswellia gum or extract which is enriched in 3-O-acetyl-11-keto-.beta.-boswellic acid (AKBA) is typical. Enriched AKBA Boswellia gums or extracts are described in PCT Patent publication WO03074063A1 which relates to a process for producing a fraction enriched up to 100% of 3-O-acetyl-11-keto-.beta.-boswellic acid. An organic solvent extract of gum resin from Boswellia species is first subjected to oxidation and then acetylation or vice versa. This converts the less potent Boswellic acids present in the fraction to AKBA. This treated fraction is subjected to further purification and separation by chromatographic separation techniques to enhance its purity and to remove contaminants there from. This process provides an access to a fraction enriched in 10-100% AKBA for therapeutic applications.

Lipid products made in accordance with the present disclosure, however, can be made very economically and can contain relatively large amounts of one or more Boswellia derivative compounds. The composition of the present disclosure, for instance, can contain one or more Boswellia derivative compounds, such as Boswellic acid, in particular 3-O-acetyl-11-keto-.beta.-boswellic acid (AKBA), in an amount greater than about 1% by weight, such as in an amount greater than about 5% by weight, such as in an amount greater than about 10% by weight, such as in an amount greater than about 15% by weight, such as in an amount greater than about 20% by weight, such as in an amount greater than about 25% by weight, such as in an amount greater than about 30% by weight. One or more Boswellia derivative compounds can be present in the composition in an amount less than about 80% by weight, such as in an amount less than about 75% by weight, such as in an amount less than about 70% by weight, based on the total weight of the lipid multiparticulate particles containing Boswellia derivative compounds.

Moreover, in one aspect, the boswellic acid is present in the composition in an amount of about 5 mg or more, such as about 10 mg or more, such as about 15 mg or more, such as about 20 mg or more, such as about 25 mg or more, such as about 30 mg or more, such as about 35 mg or more, such as about 40 mg or more, up to about 45 mg or less, or any ranges or values therebetween. Stated differently, the boswellic acid can be present in the composition such that a mammal receives about 0.05 mg/kg of body weight per day or more, such as about 0.1 mg/kg or more, such as about 0.2 mg/kg or more, such as about 0.3 mg/kg or more, such as about 0.4 mg/kg or more, such as about 0.5 mg/kg of body weight or more, such as about 0.75 mg/kg of body weight or less, or any ranges or values therebetween.

In accordance with the present disclosure, one or more Boswellia derivative compounds, such as Boswellic acid, in particular 3-O-acetyl-11-keto-.beta.-boswellic acid (AKBA), are incorporated into a liquid matrix to form multiparticulates. Examples of liquid matrices are described, for instance, in U.S. Patent Publication No. 2018/0125863, which is incorporated herein by reference. In one embodiment, the lipid matrix is different than forming micelles, microemulsions, macroemulsions, or liposomes.

The lipid matrix used to form the particles of the present disclosure, for instance, can be made from or can include many different lipid-based components, various different acid-resistant components, and the like. Examples of materials that can be used to form the liquid matrix include a fatty alcohol, a fatty acid, a fatty acid ester of a glycol and a poly glycol, a fatty acid ester of glycerol, polyglycerol, a polyglycolized glyceride, a C10-C18 triglycerides stearoyl polyoxylglyceride, a lauroyl macrogol-32 glyceride, a caprylocaproyl macrogol-8 glyceride, an oleoyl macrogol-6 glyceride, a linoleoyl macrogol-6 glyceride, myristyl alcohol, lauryl alcohol, capric alcohol, glycerol behenate, glycerol dibehenate, glycerol palmitate, hydrogenated castor oil, stearyl alcohol, behenyl alcohol, palmitic acid, stearic acid, paraffin wax, beeswax, candelilla wax, carnauba wax, polyethoxylated 12-hydroxysteric acid, a propylene glycol fatty acid ester, esterified alpha-tocopheryl polyethylene glycol succinate, a propylene glycol monolaurate (C12) ester, polyoxyl 35 castor oil, polyoxyl 40 hydrogenated castor oil, a lecithin, vitamin E, tocopheryl polyethylene glycol succinate (TPGS), a sugar fatty acid ester, a sorbitan fatty acid ester, a polyoxyethylene sorbitan fatty acid ester, a polyoxyethylene-polyoxypropylene copolymer, propylene glycol, triacetin, isopropyl myristate, diethylene glycol monoethyl ether, polyethylene glycol, glycerol, mixtures or combinations thereof.

In one embodiment, the liquid matrix is formed from at least one low flow point excipient and at least one high flow point excipient.

For example, in certain embodiments the lipid matrix may contain one or more low-flow point excipients. Low flow point excipients generally include fatty alcohols, fatty acids, fatty acid esters of glycols and poly glycols, fatty acid esters of polyglycerol and fatty acid esters of glycerol (glycerides) with flow points of less than 50° C. When the low flow point excipient is a relatively pure material, the melting point is also less than 50° C. A preferred class of low flow point excipients are low flow point glycerides. By “low flow point” excipient, such as a glyceride, is meant that the melting point of the excipient, such as a glyceride, is less than 50° C. In some embodiments, the low flow point glyceride has a melting point of less than 40° C. In some embodiments, the low-flow point excipient, such as glyceride, is a mixture of compounds, having a flow point of 50° C. or less. In some embodiments, the low-flow point excipient, such as glyceride, has a flow point of 40° C. or less. In some embodiments, the low-flow point glyceride has a low flow point of 30° C. or less. Exemplary low flow point glycerides include polyglycolized glycerides, such as some of the Gelucire products manufactured by Gattefosse, such as Gelucire® 43/01 having a nominal melting point of 43° C. Mixtures of low flow point glycerides are also effective, such as mixtures of Gelucire® 43/01 (C10-C18 triglycerides), Gelucire® 50/13 (stearoyl polyoxylglycerides), Gelucire® 44/14 (lauroyl macrogol-32 glycerides), and mixtures thereof. Other glycerides may also be used, such as fatty acid esters of glycols and poly glycols, and fatty acid esters of polyglycerols.

A function of the low flow point excipient is to ensure that at least a significant portion of the formulation matrix softens when ingested orally by a patient, at the temperature of the GI tract (about 37° C. for humans). This allows the formulation to break down by digestion in the gastro-intestinal (GI) tract, and ultimately to disperse in the GI tract to promote dissolution and absorption of the active. In certain embodiments the low flow point excipient provides a significant portion of the formulation matrix to be present in a non-crystalline liquid or amorphous state when ingested and softened in the GI tract.

Exemplary low flow point fatty alcohols include myristyl alcohol (Tm 38° C.), lauryl alcohol (Tm 23° C.) and capric alcohol (Tm 7° C.).

Exemplary low flow point fatty acids include lauric acid (Tm 44° C.) and oleic acid (Tm 16° C.).

In certain embodiments, the lipid matrix includes a high-flow point excipient. For example, in certain embodiments the lipid matrix may contain one or more high-flow point excipients. By “high flow point” excipient is meant an excipient that has a flow point 50° C. or more. High flow point excipients may also have a melting point above 50° C. High flow point excipients generally include fatty alcohols, fatty acids, fatty acid esters of glycols and poly glycols, fatty acid esters of polyglycerol, fatty acid esters of glycerol (glycerides), waxes, polar waxes and other materials with flow points of greater than 50. A preferred class of high flow point excipients are “high flow point glycerides”. By high flow point glyceride is meant that the flow point or melting point of the glyceride is 50° C. or more. In some embodiments, the high flow point glyceride has a melting point of 60° C. or more. In some embodiments, the high-melting point glyceride is a mixture of compounds, having a flow point of 50° C. or more. In some embodiments, the high-flow point glyceride has a flow point of 60° C. or more. In some embodiments, the high flow point glyceride has a flow point of 70° C. or more.

Exemplary high flow point glycerides include glycerol behenate, glycerol dibehenate, glycerol palmitate, hydrogenated castor oil, and mixtures thereof.

Often, the high flow point glyceride is a mixture of compounds that are formulated into a product and sold under a variety of trade names.

Exemplary high flow point and high melt point fatty alcohols include stearyl alcohol (Tm 58° C.) and behenyl alcohol (Tm 71° C.).

Exemplary high flow point and high melt point fatty acids include palmitic acid (Tm 63° C.) and stearic acid (Tm>70° C.).

Exemplary waxes include paraffin wax, beeswax, candelilla wax, carnauba wax, and mixtures thereof.

A function of the high flow point excipient is to aid in the manufacturability of the particles by enabling the particles to congeal at a lower temperature to obtain solid particles during the melt-spray-congeal processing. In certain embodiments the high flow point excipient aids the physical stability of the formulation. In most embodiments, the high flow point excipient is not appreciably digested in the GI tract.

In some embodiments, the lipid matrix of the particles may include other excipients to improve the performance and chemical stability of the formulations. In some embodiments, a dispersing agent is included in the particles. Exemplary dispersing agents include lecithin, glycerol monostearate, ethylene glycol palmitostearate, aluminum oxide, polyethylene alky ethers, sorbitan esters, and mixtures thereof. In one embodiment, the particles include an antioxidant to maintain chemical stability of the active agent. Exemplary antioxidants include vitamin E, tocopheryl polyethylene glycol succinate (TPGS), rosemary extract, ascorbic acid, ascorbyl palmitate, butylated hydroxyanisole (BHA), butylated hydroxytoluene (BHT), and mixtures and combinations thereof.

In some embodiments, a flow aid is used to improve the flow properties of the particles. Exemplary flow aids also known as glidants include silica, calcium silicate, cab-o-sil, silicon dioxide, calcium phosphate tribasic, colloidal silicon-dioxide, magnesium silicate, magnesium trisilicate, starch, talc, and other flow aids. In some AKBA formulations commercially available, in particular AKBA from Boswellia serrata sources are traditionally made with excipients such as celluloses or other high molecular weight polymers. These added excipients increased the viscosities of the AKBA-coating mixtures prior to encapsulation, which dictated higher than normal disc speed (RPM) to disperse the highly viscous mixtures in to microspheres. The resulting microspheres also proved to be sticky than when coating material was pure AKBA alone, and therefore required additional use of flow aids which prevent the particles from sticking together.

In one aspect, the dietary composition further contains a disintegrating agent. The disintegrating agent, for example, can be a cross-linked carboxymethyl cellulose, such as croscarmellose. Croscarmellose is a cross-linked carboxymethyl cellulose salt. In one aspect, the cross-linked carboxymethyl cellulose can be a sodium salt. In one embodiment, the cross-linked carboxymethyl cellulose can be in the form of fibers or particles. The fibers or particles can form a free-flowing powder that is typically white in color. The cross-linked carboxymethyl cellulose is hydrophilic but also insoluble. Once placed in contact with a liquid, the cross-linked carboxymethyl cellulose wicks the fluid and begins to swell. The swelling action of the cross-linked carboxymethyl cellulose causes the dietary composition to disintegrate. In this manner, the cross-linked carboxymethyl cellulose can be used to control the release of the Boswellia derivative compound.

The ability of the disintegrating agent to affect release of the Boswellia derivative compound can be controlled by controlling the type of cross-linked carboxymethyl cellulose incorporated into the composition and by controlling the amount of the disintegrating agent added to the composition. For example, the ability of the cross-linked carboxymethyl cellulose to swell can depend upon the hydration of the carboxymethyl groups by controlling the degree of substitution within the cross-linked cellulose polymer. The degree of substitution, for instance, can be greater than about 0.5, such as greater than about 0.55, such as greater than about 0.6, such as greater than about 0.65, such as greater than about 0.7, such as greater than about 0.75, such as greater than about 0.8. The degree of substitution is generally less than about 0.9, such as less than about 0.85, such as less than about 0.8, such as less than about 0.75. The degree of substitution can be determined by elemental analysis.

The amount of the disintegrating agent or the cross-linked carboxymethyl cellulose incorporated into the dietary composition can generally be greater than about 0.5% by weight, such as greater than about 1% by weight, such as greater than about 3% by weight, such as greater than about 5% by weight, And generally less than about 15% by weight, such as less than about 12% by weight, such as less than about 10% by weight, such as less than about 8% by weight.

The particles described herein are solid at ambient temperature and are generally spherical in shape. By generally spherical is meant that while most particles are essentially spherical, they do not necessarily form “perfect” spheres. Such particle variations in spherical shapes are known to those persons of ordinary skill in the art of melt-spray-congeal processing and similar particulate forming methods.

The particles may have a size ranging from an average diameter greater than about 1 μm, and generally greater that about 10 μm. Typically the particles have a size ranging from an average diameter about 40 μm to about 3000 μm, such as from about 50 μm to about 2500 μm, such as from about 80 μm to about 2000 μm, such as from about 100 μm to about 1500 μm, such as from about 200 μm to about 1000 μm, such as from about 300 μm to about 800 μm. To measure the diameters of the particulates, there are several methods that can be used, including laser diffraction, optical microscopy, and/or SEM.

In certain embodiments, the particles containing the active ingredient and lipid matrix have a flow point above 25° C., such as above 30° C., such as above 35° C., such as above 40° C.

In one embodiment, the lipid matrix composition comprises greater than 50 wt % of the low flow point excipient. In one embodiment, the lipid matrix composition comprises at least 2 wt % of the high flow point excipient. In another embodiment, the lipid matrix composition comprises less than 20 wt % of the high flow point excipient. In another embodiment the mass ratio of the low flow excipient to the high flow excipient is at least 2:1. In still another embodiment, the mass ratio of the low flow excipient to the high flow excipient is at least 3:1. In another embodiment, the mass ratio of the low flow excipient to the high flow excipient is at least 4:1. In another embodiment, the mass ratio of the low flow excipient to the high flow excipient is at least 10:1. In another embodiment, the mass ratio of the low flow excipient to the high flow excipient is at least 15:1. In another embodiment, the mass ratio of the low flow excipient to the high flow excipient is at least 20:1.

In another aspect, the lipid matrix composition contains greater than 50% by weight of one or more high flow point excipients. For example, in one embodiment, the lipid matrix is made exclusively from one or more high flow point excipients and does not contain a low flow point excipient. One or more high flow point excipients, for instance, can be present in the lipid matrix in an amount greater than about 40% by weight, such as an amount greater than about 50% by weight, such as in an amount greater than about 60% by weight, such as in an amount greater than about 65% by weight, such as in an amount greater than 70% by weight, and generally in an amount less than about 98% by weight, such as in an amount less than about 95% by weight, such as in an amount less than about 90% by weight, such as in an amount less than about 80% by weight, such as in an amount less than about 70% by weight. When greater amounts of high flow point excipients are present, one or more low flow point excipients may be present in the composition in an amount less than about 30% by weight, such as in an amount less than about 20% by weight, such as in an amount less than about 10% by weight and generally in an amount greater than 1% by weight, such as in an amount greater than about 4% by weight. The mass ratio of the high flow point excipients to the low flow point excipients can be from about 100:1 to about 1:1, such as from about 50:1 to about 10:1, such as from about 20:1 to about 5:1.

In one particular embodiment, the lipid matrix contains a wax combined with a fatty acid alcohol and a fatty acid. The wax, for instance, can comprise candelilla wax. The fatty alcohol, on the other hand, can be stearyl alcohol, while the fatty acid can be stearic acid. For example, the wax, such as candelilla wax, can be present in the composition in an amount greater than about 20% by weight, such as in an amount greater than about 25% by weight, and generally in an amount less than about 50% by weight, such as in an amount less than about 45% by weight. The fatty alcohol, on the other hand, can generally be present in an amount greater than about 10% by weight, such as in an amount greater than about 12% by weight, and generally in an amount less than about 25% by weight, such as in an amount less than about 22% by weight, such as in an amount less than about 18% by weight. The fatty acid, on the other hand, can be present in the composition in an amount greater than about 3% by weight, such as in an amount greater than about 5% by weight, such as in an amount greater than 7% by weight, and generally in an amount less than about 15% by weight, such as in an amount less than about 12% by weight, such as in an amount less than about 10% by weight.

The lipid matrix may also comprise a dispersing agent. In one embodiment, the lipid matrix is comprised of from 0 wt % to 20 wt %, such as from 0.01 wt % to 20 wt %, of a dispersing agent. In another embodiment, the lipid matrix is comprised of from 2 wt % to 10 wt % of a dispersing agent.

The lipid matrix may also comprise an antioxidant. In one embodiment, the lipid matrix comprise from 0 wt % to 20 wt %, such as from 0.01 wt % to 10 wt %, of an antioxidant. In one embodiment, the lipid matrix comprise from 1 wt % to 5 wt % of an antioxidant.

The lipid matrix may also comprise a flow aid. In one embodiment, the lipid matrix may comprise from 0 wt % to 5 wt %, such as from 0.01 wt % to 5 wt %, of a flow aid. In another embodiment, the lipid matrix may comprise from 0.5 wt % to 2 wt % of a flow aid.

The lipid matrix may also contain flavoring or sweeteners to improve the taste of the particles to the user. In one embodiment, the lipid matrix comprise from 0 wt % to 15 wt %, such as from 0.01 wt % to 10 wt %, of an flavoring or sweetener. In one embodiment, the lipid matrix comprise from 1 wt % to 5 wt % of an antioxidant flavoring or sweetener. Flavoring and sweeteners include essential oils other sweeteners used in the nutraceutical or food industries.

The lipid matrix described herein may be formulated by any suitable process. In some embodiments, the matrix may be formulated by a suitable melt-spray-congeal process.

A molten mixture is formed by mixing and heating the lipid matrix compositions as previously described. “Molten mixture” means that the mixture of an active ingredient and lipid matrix materials are sufficiently mixed and heated to fluidize the mixture sufficiently to allow it to be atomized into droplets. Generally, the mixture is molten in the sense that it will flow when subjected to one or more forces such as pressure, shear, and centrifugal force, such as that exerted by a centrifugal or spinning-disk atomizer.

Once the molten mixture has been formed, it is delivered to an atomizer that breaks the molten mixture into small droplets. Virtually any method can be used to deliver the molten mixture to the atomizer. In certain embodiments of the disclosed methods the molten mixture is delivered to the atomizer by use of pumps and/or various types of pneumatic devices such as pressurized vessels or piston pots or extruder. In certain embodiments the molten mixture is maintained at an elevated temperature during delivery to the atomizer to prevent its solidification and to keep it in a flowable state.

When a centrifugal atomizer (also known as rotary atomizers or spinning-disk atomizer) is used, the molten mixture is fed onto a rotating surface, where it spreads outward and flows by centrifugal force. The rotating surface may take several forms, examples of which include a flat disk, a cup, a vanned disk, and a slotted wheel. The surface of the disk may also be heated to aid in atomization of the molten mixture or cooled to aid in the solidification of the cores containing the lipid matrix. Several mechanisms of atomization are observed with flat-disk and cup centrifugal atomizers, depending on the flow of molten mixture to the disk, the rotation speed of the disk, the diameter of the disk, the viscosity of the feed, and the surface tension and density of the feed. At low flow rates, the molten mixture spreads out across the surface of the disk and when it reaches the edge of the disk, forms a discrete droplet, which is then flung from the disk.

Once the molten mixture has been atomized, the droplets are congealed, typically by contact with a gas at a temperature below the solidification temperature of the composition. Typically, it is desirable that the droplets are congealed in less than 60 seconds, less than 10 seconds, or even in less than 1 second. In certain embodiments congealing at ambient temperature using an ambient temperature cooling medium, results in sufficiently rapid solidification of the droplets. However, as certain embodiments of the disclosed compositions are comprised of at least 50 wt % of a low flow point excipient, it is often preferred to utilize a cooling medium that is at a temperature that is at least 10° C. below ambient temperature. For some embodiments, it is preferred to utilize a cooling medium that is at least 20° C. below ambient temperature.

In one aspect, one or more surfactants can optionally be incorporated into the composition. Surfactants can be incorporated into the composition for various reasons. It was discovered that some surfactants can actually facilitate control of the delayed release function of the composition. In some embodiments, surfactants and co-surfactants may be included in the compositions. Exemplary surfactants and co-surfactants include polyethoxylated 12-hydroxysteric acid, also known as PEG15 hydroxy stearate (Kolliphor® HS-15), propylene glycol monocaprylate (C8) esters (Caproyl™ 90), esterified alpha-tocopheryl polyethylene glycol succinate (TPGS), mono, di, tricaprylic (C8) and capric acid (C10) esters of glycerol and mono and diesters of PEG400 (Labrasol®), Propylene glycol monolaurate (C12) esters (Labrafil® M1944CS), Polyoxyl 40 hydrogenated castor oil (Kolliphor® RH40), lecithins, and mixtures thereof.

In one embodiment, the surfactant incorporated into the composition can be a polysorbate, a sulfate surfactant, or mixtures thereof. Sulfate surfactants include, for instance, salts of fatty acids sulfates. For example, in one embodiment, the surfactant can be sodium laureth sulfate.

The amounts of surfactants incorporated into the composition can vary widely depending upon the reason for adding the surfactant or the desired result. In general, when included in the composition, one or more surfactants can be present in an amount greater than about 1% by weight, such as in an amount greater than about 3% by weight, such as in an amount greater than about 7% by weight, such as in an amount greater than about 10% by weight, such as in an amount greater than about 15% by weight, such as in an amount greater than about 20% by weight, such as in an amount greater than about 25% by weight, such as in an amount greater than about 30% by weight. One or more surfactants are generally present in the composition in an amount less than about 50% by weight, such as in an amount less than about 40% by weight, such as in an amount less than about 30% by weight, such as in an amount less than about 20% by weight, such as in an amount less than about 10% by weight.

Further advantages of encapsulating the Boswellia derivative compound in the lipid multiparticulate include that the resulting lipid multiparticulates are stable and neutral tasting lipid. This is an advantage since Boswellia derivative compound are typically odiferous which can result in some users rejecting these Boswellia compounds as nutraceuticals. In addition, the lipid multiparticulates of Boswellia derivative compounds, such as AKBA, release the Boswellia derivative compounds, including AKBA, over a period of time once ingested. This will provide a lasting benefit of the Boswellia derivative compound providing an anti-inflammatory benefit to the use over a longer period of time. Further, the lipid multiparticulates may further increase the bioavailability of the Boswellia derivative compounds.

Other benefits of the lipid multiparticulates is that the Boswellia derivative compounds, including AKBA, can be in products such as nutritional bars; and in sachet formats for adding in to oatmeal, cereals, ready-to-mix (RTM) type beverages, salads, and other similar food products to achieve the benefits of the Boswellia derivative compounds, including AKBA.

In some embodiments, the one or more particles provided herein may be formulated into any suitable dosage formulation. For example, in certain embodiments, the one or more particles provided herein may be placed into a capsule for delivery by oral ingestion. Exemplary capsules include hard gelatin capsules, soft gelatin capsules, HPMC capsules, as well as capsules made from other materials. The one or more particles may be suspended in an aqueous-based matrix or an oil-based matrix within the capsule itself. In certain embodiments where the particles are suspended in an aqueous-based matrix or an oil-based matrix, the aqueous-based matrix or oil-based matrix may additionally include one or more active ingredients. In certain embodiments, the one or more particles may be contained within a monolithic enteric capsule suitable for providing a modified release profile when ingested.

Capsules normally include a shell filled with one or more specific substances. The shell itself may be a soft or a hard capsule shell. Hard capsule shells are generally manufactured using dip molding processes, which can be distinguished into two alternative procedures. In the first procedure, capsules are prepared by dipping stainless-steel mold pins into a solution of polymer, optionally containing one or more gelling agents (e.g. carrageenan) and co-gelling agents (e.g. inorganic cations). The mold pins are subsequently removed, inverted, and dried to form a film on the surface. The dried capsule films are then removed from the molds, cut to the desired length, and then the telescoping fit caps and bodies are assembled together, printed, and packaged. In the second procedure, no gelling agents or co-gelling agents are used and film-forming polymer solution gelification on the molding pins is thermally induced by dipping pre-heated molding pins into the polymer solution. This second process is commonly referred to as thermogellation, or thermogelling dip molding. The aforementioned manufacturing processes involve the use of solutions of the different ingredients that are needed for the making the telescoping fit hard capsule shells.

Hard capsules may be filled with active ingredients, such as the particles described herein, via procedures known in the art. Typically, active ingredients are combined with various compatible excipients for ease of fill. The resulting fill may be a dry powder, a granulation, particles, lipid particles, a suspension, or a liquid. Additionally, stable, filled hard capsules have advantages over other dosage delivery forms such as liquids and solid tablets. Certain active ingredients may be difficult to formulate into dry granules or may be otherwise incompatible with the tableting process. Another consideration is improved patient compliance for taste-masking and ease of swallowing, i.e., capsules being preferred by consumers over tablets. For example, in some embodiments, provided is a pharmaceutical composition that contains a capsule filled with the one or more particles disclosed herein. In some embodiments, the one or more particles have not been enterically coated for modified release or gastric protection.

In certain other embodiments, the one or more particles can be administered orally as a solid, liquid, suspension, or other suitable delivery means. The composition of particles may be administered via buccal or sublingual administration. In one embodiment, the one or more particles may be administered as a capsule, tablet, caplet, pill, troche, drop, lozenge, powder, granule, syrup, tea, drink, thin film, seed, paste, herb, botanical, and the like.

In a further embodiment of the present disclosure, the lipid multiparticulate particles described herein can be combined with or used with other nutraceutical components to form a nutraceutical composition. The lipid multiparticulates of Boswellia derivative compound, in particular AKBA, can be blended with other nutraceutical components which result in stable combinations of lipid multiparticulates of AKBA and other nutraceutical ingredients in both nutraceutical finished solid and liquid dosages, as well as in food and beverage applications. Exemplary nutraceuticals which can be blended with the lipid multiparticulates include the collagen, including hydrolyzed collagen or undenatured collagen, including but not limited to UC-II® product available from Lonza, probiotics, for example, but not limited to TWK10® product available from Lonza, enzymes, endogenous fatty acid amides, cetylated fatty acid esters, omega-3 fatty acids, hyaluronic acids, curcuminoids, herbal and botanical extracts, carotenoids, methylsulfonylmethane (MSM), carnitine, including but not limited to, Carnipure® available from Lonza, and antioxidants, for example, Oceanix™ available from Lonza. Other nutraceutical ingredients having anti-inflammatory benefits such as turmeric curcuminoids, eggshell membrane, green lipped mussel, omegas-3 EPA and DHA, krill oil, French maritime pine bark extract (Pycnogenol®), Scutellaria baicalensis and Acacia catechu extracts (Univestin®), ashwagandha extract, rose hip extract, tart cherry extract, astaxanthin, hops extract (Perluxan®), glucosamine, chondroitin, hyaluronic acid, salmon nasal cartilage, avocado soy unsaponifiable, methylsulfonylmethane (MSM), willow bark extract, tamarind seed extract, lactobacillus and bifidobacteria probiotic strains (e.g. TWK10@product available from Lonza), palmitoylethanolamide (PEA), and cetyl myristoleate (CM), which may further eliciting anti-inflammation health benefits.

In the present disclosure, also provided is method for administering a Boswellia derived compound to a mammal over an extended period of time. The method includes orally administering to a mammal an extended release composition comprising lipid multiparticulate particles, the lipid multiparticulate particles comprising a lipid matrix and wherein dispersed in the lipid matrix is an active agent, the active agent comprising a Boswellia derived compound. In a typical dosage, the Boswellia derived compound is typically administered to the mammal containing the in an amount from about 1 mg to about 1,000 mg, for example, 2 mg to about 500 mg and more particularly between about 5 mg to 200 mg. Depending on the percentage of the Boswellia derivative compound in the lipid multiparticulate, the amount of the lipid multiparticulate is adjusted to achieve the correct dosage. In addition, the AKBA-LMP may also exhibit a delayed release of the AKBA, such as to avoid release of the AKBA in the stomach of a user.

Nonetheless, certain embodiments of the present disclosure may be better understood according to the following examples, which are intended to be non-limiting and exemplary in nature.

### Example 1

A formulation was prepared from a Boswellia extract material with a known concentration of boswellic acids. Specifically, Boswellia serrata extract material in 48% (w/w) compositions was mixed and agitated constantly in to molten candelilla wax (48% w/w) and the temperature was kept at 70-75° C. until Boswellia extract was completely suspended in waxes. The resulting glossy molasses-like mixture was added with about 4% (w/w) stearyl alcohol and was heated at the same temperature for a period of time until stearyl alcohol was completely melted and suspended. The final mixtures were the processed in a melt spray congeal process, producing microparticles of 200 microns. High performance liquid chromatography (HPLC) analyses of the microparticles showed that boswellic acids were coated in candelilla waxes, and survived heating and spinning processes, and was qualitatively assayed to contain ˜48% boswellic acids using starting Boswellia serrata extract material as reference.

### Example 2

In another preparation, Boswellia serrata extract material (˜66% w/w) was mixed and constantly agitated in to molten candelilla waxes (˜33% w/w) until glossy molasses-like mixtures were obtained. In these mixtures about 1% (w/w) stearyl alcohol powders were mixed in with constant agitation at 70-75° C. to melt and suspend stearyl alcohol. Likewise, the resulting mixtures were processed in a melt spray congeal process, producing microparticles of at least 100 microns particle sizes. Following same HPLC analyses for Example 1 above, lipid microparticles of Example 2 showed presence and levels of boswellic acids at about 73% in these preparations.

### Example 3

Four formulations of AKBA LMP were prepared and are labeled as Formulations 3, 4, 5 and 6 so as not be confused with examples 1 and 2.

### Formulation 3

A preparation with Boswellia serrata extract materials with at least 50% 3-O-acetyl-11-keto-.beta.-boswellic Acid (AKBA) were formulated at about 44 w/w in molten mixtures of equal amounts candelilla wax and stearic acid between temperatures of 70-75° C. in accordance with the process describe in Example 1. After achieving a homogeneous mixture, the completely suspended Boswellia serrata extracts were converted into lipid microparticles using a melt spray congealing unit and yield microparticles at sizes 300 to 600 microns. HPLC analyses from these mixtures reported at least 22% AKBA.

### Formulation 4

Another preparation, Boswellia serrata extract materials high in 3-O-acetyl-11-keto-.beta.-boswellic Acid (AKBA) content were formulated at about 44% w/w levels in molten mixtures of candelilla wax (˜18% w/w) and stearic acid (˜38% w/w) at temperatures of 70-75° C. These molten mixtures were processed in melt spray congeal units, and microparticle sizes from 300 microns to 600 microns were obtained. Analytical testing showed that AKBA contents from lipid multiparticulates were at least 22% AKBA levels.

### Formulation 5

In a separate preparation, Boswellia serrata extract materials standardized with high 3-O-acetyl-11-keto-.beta.-boswellic Acid (minimum of 50% AKBA) were formulated at about 44% w/w levels, and were initially mixed with molten mixtures of candelilla wax (˜20.5% w/w) and stearic acid (˜30.5% w/w) at 70-75° C. The resulting mixtures were added with sunflower lecithin with 60% phospholipids, at 5% w/w levels and the final resulting mixtures were mixed and kept at 75° C., before these molten mixtures were sprayed in melt spray congeal units. Microparticle sizes from 300 microns to 600 microns were also obtained, and analytical testing showed that AKBA contents from lipid multiparticulates were at 22% AKBA minimum.

### Formulation 6

Another formulation was prepared made using Boswellia serrata extract material with 3-O-acetyl-11-keto-.beta.-boswellic Acid (minimum of 50% AKBA) at about 44% w/w in molten mixtures of both candelilla wax and stearic acid, at 12.7% w/w, and 43% w/w, respectively. The final mixtures were kept at 70-75° C. until the extract materials were completely dispersed and suspended in molten lipids. After melt spraying processed, lipid microparticles of 300-600 microns were obtained. Analytical testing showed a minimum of 22% AKABA from these mixtures.

The lipid multiparticulates of Boswellia derivative compound, primarily 3-O-acetyl-11-keto-.beta.-boswellic Acid (AKBA) shown as Formulations 3, 4, 5 and 6, were tested for extended release profiles when particles underwent dissolution studies at initially in acid pH 0.1 media, then followed immediately by buffered media at pH 6.8 with sodium lauryl sulfate (SLS). Small volume aliquots were pulled from respective dissolution baths at each studied time point (1-hr, 2-hr, 4-hr, 6-hr, 8-hr, 10-hr, 12-hr, 16-hr, and 24-hr), and were analyzed accordingly using established and previously reported methodologies in literature. The Figure illustrates extended release dissolution profiles for AKBA from different lipid multiparticulate formulations, with 0% AKBA released from 1-hr to 2-hr dissolution (delayed-release), and started releasing AKBA from lipid multiparticulates into the buffered media, from 4-hr (20%-40% release) until 80%-100% AKBA release, from 10-hr to 24-hr dissolution (extended-release). Also, the dissolution studies clearly showed the protective effects of lipid multiparticulate encapsulation for AKBA, while incrementally releasing AKBA over 24-hr period.

### Example 4

A further example was conducted according to the following procedure. Namely, the efficacy of LMP-AKBA (LMP formulated 3-O-Acetyl-11-keto-β-boswellic acid) formulations in a monosodium iodoacetate (MIA)-induced knee OA model in rats were assessed.

A total of 35 female Sprague Dawley rats (age: 8 weeks, mean weight: 180±20 g) purchased from Firat University Experimental Research Center (FUDAM) were used in the study. Throughout the experiment, the rats were kept in a room with controlled temperature (23±2° C.), humidity (55±10%), with light (12/12 h light-dark cycle) and food/water ad libitum. The study was approved by the Animal Ethics Committee of Firat University and done in accordance with the standard ethical guidelines for laboratory animal use rules.

### Experimental Design

Female Sprague Dawley rats (age: 8 weeks) were randomly allocated into groups (n=7 in each):
1. Control—(No OA disease/No treatment)
2. Monosodium iodoacetate (MIA) Control—(Yes OA disease/No treatment)
3. MIA+LMP AKBA—(100 mg/kg BW HED*; low dose)
4. MIA+LMP AKBA—(200 mg/kg BW HED*; high dose)
5. MIA+Unformulated AKBA—(200 mg/kg BW HED*)

TABLE 1

| | | | Animal | Active, mg | HED, mg |
|---|---|---|---|---|---|
| Group | Treatments | Lot # | Dose | (HED) | (60 kg BW) |
| 1 | — | — | — | — | — |
| 2 | MIA | — | — | — | — |
| | (1 mg/kg BW) | | | | |
| 3 | MIA + LAK 1 | lot JB-082321- | 10.3 mg/ | 22 mg | 100 mg |
| | (22% AKBA) | F1222 | kg BW | AKBA | |
| 4 | MIA + LAK 2 | lot JB-082321- | 20.6 mg/ | 44 mg | 200 mg |
| | (22% AKBA) | F1222 | kg BW | AKBA | |
| 5 | MIA + UAK | lot UO/BSD | 8.5 mg/ | 44 mg | 82 mg |
| | (53.4% AKBA) | 1610/0521/22 | kg BW | AKBA | |

*LMP = lipid multiparticulate;
HED = human equivalent dose.

The OA rat models were performed as previously described (Lu et al., 2018, Jeong et al., 2017). In order to induce OA rat model, the right knee of the rats was shaved and disinfected with 70% alcohol following anaesthetization using with xylazine (10 mg/kg) and ketamine hydrochloride (50 mg/kg). 3 mg of MIA (Sigma, St. Louis, USA) were dissolved in 50 μL saline and injected into right knee joints through the infrapatellar ligament using a 0.3 ml insulin syringe fitted with a 29-G needle. Control group (Group 1) received an injection of 50 μL saline. Two weeks after injection with MIA, the LMP AKBA and ULAN1 samples were dissolved in 1 mL saline and orally given for 4 weeks (total study duration was 6 weeks). All rats were observed every other alternate day to assess knee joint swelling. Four weeks after administering the control and samples, the rats were sacrificed, and blood and the specimens of the knee joint were collected for the follow-up experiment. The blood samples were centrifuged at 3,000 rpm for 10 min, and the harvested sera were kept at −20° C. until the day of analysis.

### X-Ray Analyses

Experienced senior radiologists determined the severity of OA in all rats. The severity in each joint was evaluated according to the Kellgren-Lawrence scoring system. In the scoring system, each radiograph was assigned a grade from 0 to 4 in correlation with the severity of OA, grade 0 signifying no presence of OA and Grade 4 signifying severe OA.

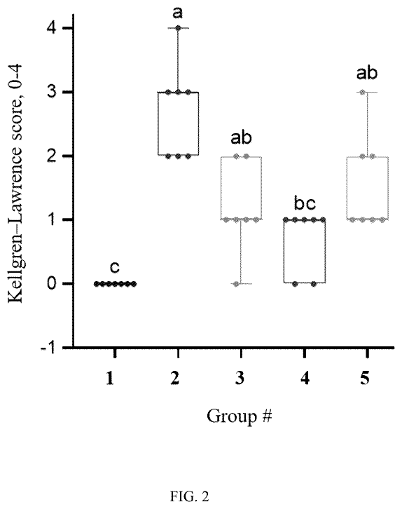

The results are illustrated in FIG. 2. Bars with different letters are significantly different. The results depicted in FIG. 2 show reduced radiographic score in treatment Groups 3-5, which suggests a decrease in OA severity in treatment Groups 3-5 as compared to the disease model control without treatment in Group 2. Unexpectedly, decrease in OA severity in treatment Group 4 was the most notable.

### Behavioral Tests

For this purpose, direct pain behavior performance (for detecting weight-bearing asymmetry) and indirect pain behavior performance (for detecting allodynia) were tested. Behavioral evaluations were performed twice a week for each rat from the week before local injection. All data were collected and applied for statistical at 0, 7, 14, 21, and 28 days. Weight-bearing changes in the hind paw represented the weight distribution between the right (operated) and left (control) limbs as a direct index of joint pain in the osteoarthritic knee.

A von Frey test was used to measure the mechanical threshold for indicating allodynia, which was induced by mechanical stimulation. Rats were placed in a chamber with a mesh bottom, which allowed access to the plantar surface of each hind paw. The animals were allowed to acclimatize in the chamber for 10 min before testing. The mechanical threshold of the ipsilateral hind paw were assessed using the modified up-down method. A von Frey hair were perpendicularly applied to the plantar surface of the ipsilateral hind paw until the hair flexed and held in place for 3 s.

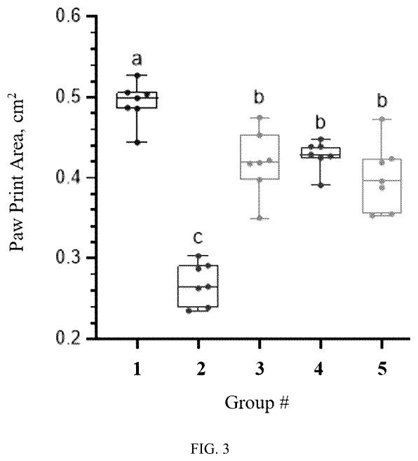

The results are illustrated in FIG. 3. Increase in paw print area suggests improved weight bearing, which is indirectly corelated to reduced pain. Bars with different letters in FIG. 3 are significantly different. Paw print areas in treatment Groups 3-5 were significatly increased over Group 2 (OA disease without treatment). The results of the von Frey test indicate outcomes related to hind paw withdrawal mechanical thresholds. The results depicted in FIG. 3 show increase in paw print area (reduction in pain sensitivity) in treatment Groups 3-5, which indirectly correlates to a reduction in pain in treatment Groups 3-5 as compared to the disease model control without treatment in Group 2.

### Joint Swelling (Edema)

Three measures of knee joint thickness (knee diameter) were taken under anesthesia (02: 2.0 L/m, 2% isoflurane), using an electronic digital caliper (Mitutoyo Absolute Digimatic 150 mm, Japan). The results were expressed in mm.

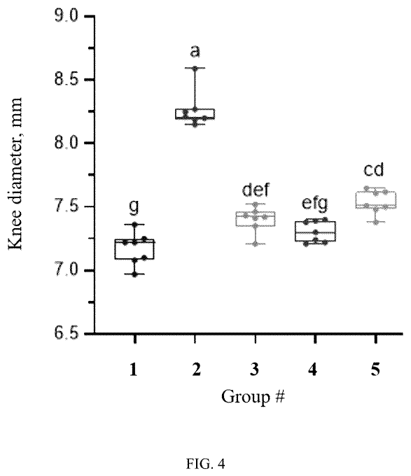

The results are illustrated in FIG. 4. Reduced knee diameter suggests reduced knee swelling. Bars with different letters are significantly different. The results depicted in FIG. 4 show reduction in knee diameter in treatment Groups 3-5, which correlates to a reduction in knee swelling in treatment Groups 3-5 as compared to the disease model control without treatment in Group 2.

### Biochemical Analysis

At the end of the study, the rats were sacrificed, and blood samples were collected. The blood samples were centrifuged, and the collected sera were kept at −80° C. Serum biochemical parameters, namely blood urea nitrogen (BUN), and creatine levels, as well as alanine transaminase (ALT) and aspartate aminotransferase (AST) activities, were assessed biochemistry analyzer (Samsung Electronics Co., Suwon, Korea). Enzyme-linked immunosorbent assay (ELISA) kits (Cayman Chemical, Ann Arbor, MI, USA) were used in analyzing serum inflammation parameters of IL-1β, IL-6, TNF-α, and C-reactive protein (CRP), the cartilage degeneration mediators cartilage oligomeric matrix protein (COMP) and C-telopeptide of type II collagen (CTX-II) according to the manufacturer instructions.

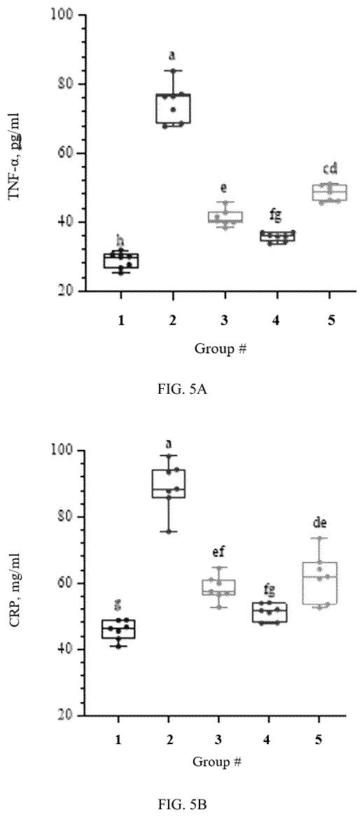

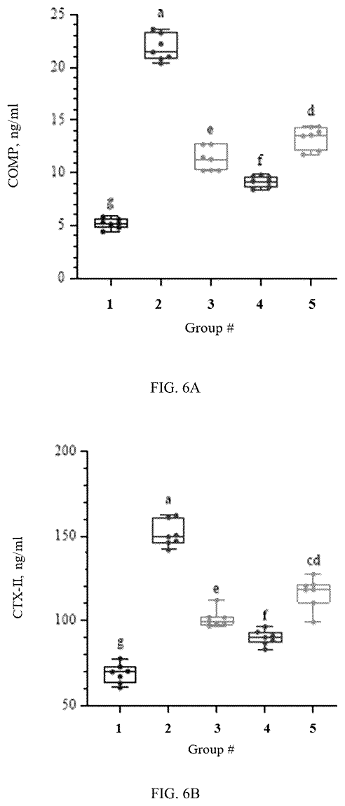

The results related to inflammatory biomarkers are illustrated in FIGS. 5A and 5B. The results related to cartilage breakdown biomarkers are illustrated in in FIGS. 6A and 6B. Bars with different letters are significantly different. Reduced levels of IL-6, TNF-α, CRP, COMP, LOX-5, NF-κB, and TGF-1β were observed. Decrease in TNF-α and CRP suggests a decrease in inflammation (FIGS. 5A and 5B). Decrease in COMP and CTX-II suggests a decrease in cartilage breakdown (FIGS. 6A and 6B). Overall, the results suggest anti-inflammatory action and reduced cartilage damage in treatment Groups 3-5 as compared to the disease model control without treatment in Group 2.

The present inventors have surprisingly found that, in the OA animal model, treatment Group 3 (MIA+LMP AKBA—(100 mg/kg BW HED; low dose) and treatment Group 4 (MIA+LMP AKBA—(200 mg/kg BW HED; high dose), most notably, exhibited reduced pain, increased weight bearing, reduced knee swelling, reduced cartilage damage, and reduced inflammatory markers. Group 4, in particular, showed unexpectedly positive improvements in joint health. The results are summarized in Table 2.

TABLE 2

| | | ↑ Weight | | ↓ OA | | |
|---|---|---|---|---|---|---|
| | | bearing | ↓ Knee | Disease | | |
| | ↓ Pain | (% Paw | Swelling | Severity | ↓ Cartilage | ↓ Inflammatory |
| Group # | (%)* | Print)* | (%)* | (%)* | damage | markers |
| 3 | +++ | +++ | +++ | +++ | +++ | +++ |
| 4 | ++++ | ++++ | ++++ | ++++ | ++++ | ++++ |
| | (67%) | (70%) | (12%) | (74%) | | |
| 5 | + | + | + | + | + | ++ |

*Percent change in different parameters versus Group 2 (i.e., the control OA group).

Surprisingly, animal OA models in treatment Group 4 showed 67% reduction in pain versus control OA group, 70% increase in weight bearing versus control OA group, 12% reduction in knee swelling versus control OA group, 74% reduction in OA disease severity versus control OA group, as well as significant reduction in inflammatory and cartilage damage biomarkers versus control OA group. Unexpectedly, it was found that improved joint health can be achieved by the present disclosure.

### Statistical Analysis

The samples size of the study was determined to be totally 35 rats (n=7) with the help of G*Power package program (Version 3.1.9.2) with alpha error 0.05 and 90% power with effect size 0.80 calculated. SPPS statistical package program (IBM SPSS Version 22.0) was used to estimate the data. In this study, conformity to the assumption of normality from the prerequisites of the parametric tests was performed using the “Shapiro-Wilk” test and the homogeneity of the variances were checked with the “Levene” test. Analysis of variance (ANOVA) test was performed to determine the differences between the groups and post-hoc Tukey test used for multiple comparisons of the groups. Radiologic and histopathologic scores, nonparametric data, were analyzed using Kruskal-Wallis followed by Mann-Whitney U. Statistical significance was accepted as P<0.05.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

What is claimed:

1. An extended release composition comprising:
lipid multiparticulate particles, and
an active agent,
the lipid multiparticulate particles comprising a lipid matrix, and wherein the active agent is dispersed within the lipid matrix, the active agent comprising a Boswellia derived compound, wherein the active agent is released from the lipid multiparticulate particles over a period of time.

2. The extended release composition as defined in claim 1, wherein the active agent comprises a Boswellic acid compound.

3. The extended release composition as defined in claim 2, wherein the Boswellic acid compound comprises 3-O-acetyl-11-keto-.beta.-boswellic acid (AKBA).

4. The extended release composition as defined in claim 1, wherein the active agent is released from the composition over a period up to about 30 hours after ingestion by a mammal.

5. The extended release composition as defined in claim 1, wherein the active agent is encapsulated by the lipid matrix.

6. The extended release composition as defined in claim 1, wherein the active agent is present in the lipid multiparticulate particles in an amount from about 1% to about 80% by weight based on a total weight of the lipid multiparticulate particles.

7. The extended release composition as defined in claim 1, wherein the extended release composition is in a form of a capsule, a tablet, a powder, or is suspended in a liquid.

8. The extended release composition as defined in claim 1, wherein the lipid multiparticulate particles have an average particle size of from about 40 microns to about 3000 microns.

9. The extended release composition as defined in claim 1, wherein the lipid matrix comprises at least one low flow point excipient and at least one high flow point excipient.

10. The extended release composition as defined in claim 1, wherein the lipid matrix comprise a fatty alcohol, a fatty acid, a fatty acid ester of a glycol and a poly glycol, a fatty acid ester of glycerol, polyglycerol, a polyglycolized glyceride, a C₁₀-C₁₂ triglyceridesstearoyl polyoxylglyceride, a lauroyl macrogol-32 glyceride, a caprylocaproyl macrogol-8 glyceride, an oleoyl macrogol-6 glyceride, a linoleoyl macrogol-6 glyceride, myristyl alcohol, lauryl alcohol, capric alcohol, glycerol behenate, glycerol dibehenate, glycerol palmitate, hydrogenated castor oil, stearyl alcohol, behenyl alcohol, palmitic acid, stearic acid, paraffin wax, beeswax, candelilla wax, carnauba wax, polyethoxylated 12-hydroxysteric acid, a propylene glycol fatty acid ester, esterified alpha-tocopheryl polyethylene glycol succinate, a propylene glycol monolaurate (C₁₂) ester, polyoxyl 35 castor oil, polyoxyl 40 hydrogenated castor oil, a lecithin, vitamin E, tocopheryl polyethylene glycol succinate (TPGS), a sugar fatty acid ester, a sorbitan fatty acid ester, a polyoxyethylene sorbitan fatty acid ester, a polyoxyethylene-polyoxypropylene copolymer, rosemary extract, propylene glycol, triacetin, isopropyl myristate, diethylene glycol monoethyl ether, polyethylene glycol, glycerol, or mixtures or combinations thereof.

11. The extended release composition as defined in claim 1, wherein the lipid matrix comprises a wax, a fatty alcohol, and a fatty acid.

12. The extended release composition as defined in claim 11, wherein the wax comprises candelilla wax, wherein the fatty alcohol comprises stearyl alcohol, and wherein the fatty acid comprises stearic acid.

13. The extended release composition as defined in claim 1, wherein the lipid matrix further comprises
(a) a surfactant, or
(b) a cross-linked carboxymethyl cellulose salt.

14. The extended release composition as defined in claim 13, wherein the surfactant comprises a polysorbate, a laureth sulfate, or mixtures thereof.

15. The extended release composition as defined in claim 9, wherein the low flow point excipients are present in the composition in an amount of from about 0.1% to about 20% by weight and wherein the high flow point excipients are present in the composition in an amount of from about 30% to about 85% by weight based on a total weight of the composition.

16. (canceled)

17. The extended release composition as defined in claim 1, further comprising flow aids, antioxidant, dispersing agent and/or a flavoring or sweetener.

18. A method for administering a Boswellia derived compound to a mammal over an extended period of time, said method comprising:
orally administering to a mammal an extended release composition comprising lipid multiparticulate particles, the lipid multiparticulate particles comprising a lipid matrix and wherein dispersed in the lipid matrix is an active agent, the active agent comprising a Boswellia derived compound, each dosage administered to the mammal containing the Boswellia derived compound in an amount from about 1 mg to about 1,000 mg.

19. A method as defined in claim 18, wherein the extended release composition is formulated such that Boswellia derived active agent is released from the extended release composition in period of time from about 0.5 hours to 24 hours after administration to a mammal.

20. A method as defined in claim 18, wherein the Boswellia derived compound comprising 3-O-acetyl-11-keto-.beta.-boswellic acid (AKBA).

21. A nutraceutical composition comprising the extended release composition according to claim 1 and a second nutraceutical ingredient.

22. The nutraceutical composition according to claim 21, wherein the second nutraceutical ingredient comprises undenatured collagen.

23. A method of reducing inflammation in a mammal, said method comprising providing the extended release composition of claim 1 and administering the extended release composition to the mammal in need of a reduction in inflammation.

24. A method of increasing bioavailability of a Boswellia derived compound in a mammal said method comprises forming an extended release composition according to claim 1 and administering the extended release composition to the mammal.
